(19) United States
(12) Patent Application Publication
(10) Pub. No.: US 2026/0248733 A1
(43) Pub. Date: Aug. 27, 2026

(54) NEMATOCYSTS AS NATURAL MICROINJECTORS

(71) Applicant: Battelle Memorial Institute, Columbus, OH (US)

(72) Inventors: Jacob Beaver, Columbus, OH (US); Emily Breech, South Bloomfield, OH (US); Vic Simons, Powell, OH (US); Dave Albertson, Dublin, OH (US); Sarah Pruitt, Columbus, OH (US); Sarah Ducceschi, Hilliard, OH (US); Megan Cruz, Medina, OH (US)

(21) Appl. No.: 18/875,800

(22) PCT Filed: Jun. 28, 2023
(86) PCT No.: PCT/US23/69216

§ 371 Date: Dec. 17, 2024

Related U.S. Application Data
(60) Provisional application No. 63/356,552, filed on Jun. 29, 2022.

(51) Int. Cl. A61K 9/50; A61K 9/06; A61K 9/51; A61K 47/32; A61K 47/34

(52) U.S. Cl. CPC A61K 9/5068; A61K 9/06; A61K 9/5192; A61K 47/32; A61K 47/34

(57) ABSTRACT

The present invention is directed at the formation of repurposed nematocysts which can then provide a transdermal or cutaneous delivery system.

### CROSS-REFERENCE TO PRIOR APPLICATIONS

This application claims priority to and the benefit of U.S. Provisional Application No. 63/356,552 filed Jun. 29, 2022, the entirety of which is incorporated by reference.

### FIELD

The present invention is directed at the formation of repurposed nematocysts which can then provide a transdermal or cutaneous delivery system.

### BACKGROUND

Jellyfish and sea anemones rely upon stinging cells in their tenacles and digestive capacity to catch food. The cellular components of these stinging cells are known as cnidocytes or nematocytes. More specifically, a cnidocyte or nematocyte is reported as an explosive cell containing a relatively large secretory organelle or subunit of the cell called a cnidocyst or nematocyst) that can deliver a sting to other organisms.

### SUMMARY

A method of providing nematocysts for a transdermal or cutaneous delivery comprising: providing nematocysts sourced from the genus Cassiopea or Chrysaora containing enodgenous toxins, force-firing the nematocysts and collecting the endogenous toxins and treating the nematocysts with an enzyme and inactivating the endogenous toxins. The nematocysts may then be loaded with a therapeutic agent.

A method of providing nematocysts for a transdermal or cutaneous delivery comprising providing nematocysts sourced from the genus Cassiopea or Chrysaora containing enodgenous toxins, treating the nematocysts with an enzyme configured to inactivate the endogenous toxins and force-firing the nematocysts and inactivating said endogenous toxins. The nematocysts may then be loaded with a therapeutic agent.

A pharmaceutical composition comprising as an active ingredient, a therapeutic agent loaded into nematocysts sourced from the genus Cassiopea or Chrysaora.

### DETAILED DESCRIPTION OF THE PREFERRED EMBODIMENTS

Nematocytes are repurposed to provide for a transdermal or cutaneous delivery system. Such an injection system is contemplated to be an eco-friendly alternative to needle-based delivery systems for humans and animals. The delivery systems herein are also contemplated to provide a relatively painless injection system for delivery of therapeutics and prophylactics into the skin, along with the ability to dissolve into the skin and not produce medical device waste.

The nematocytes are preferably and initially isolated and then preferably configured to be neutralized by removal of residual toxins. Preferably, it is contemplated that such neutralization may occur by forcing the isolated nematocytes to release their toxic venom. This is contemplated to occur by force-firing isolated nematocysts to collect cnidome proteins, which then are preferably treated with a serine protease. Nematocysts can be forced to fire by treatment with a hypotonic solution (i.e. distilled water). The firing mechanism of the nematocyst relies on an osmotic gradient. The internal area of the nematocyst is highly hypertonic to seawater. Thus, when the cnidocil is stimulated and the opercula opens, water enters the caps rapidly and releases the hydrostatic pressure inside. Examples of such serine protease include chymotrypsin, trypsin, and elastase. In addition, nematocysts may be treated with serine protease and then force-fired and cnidome is collected. Treatment with a serine protease (or a proteinase cocktail including collagenase, peptidase K, and papain-like protease) can successfully inactivate and digest any endogenous toxins present in the nematocyst.

Nematocysts herein are preferably collected from the genus Cassiopea and Chrysaora. Nematocysts isolated from Chrysaora will be specifically A-isorhiza, O-isorhiza, and macro/micro mastigophores. These are preferential due to their near spherical shape and their ability to discharge their filaments at a length of 200 μm to 500 μm. Cassiopea animals are of particular interest here due to their unique ability to produce relatively small bodies, having a size of 0.1 mm to 5.0 cm, called cassiosomes, containing the nematocytes. These structures are relatively small, amorphous, hollow clusters of cnidocytes that are secreted in mucus upon agitation, stimulation, or in the presence of potential food. The generation of cassiosomes provides a unique purification where specific types of nematocysts (namely O-izorhiza) are selected to populate the structure. Collection of nematocysts from the cassiosomes allows for the acquisition of a homogenous nematocyst suspension, rather than a mixed population. By way of working example, one can condense 15 mls of Cassiopea mucus into a solution of disassociated nematocysts at a concentration of 1×106 nematocysts per 1 ml. This solution is 100% O-isorhiza morphology.

Worthy of note is that in the case of the Chrysaora jellies one may take snips of the tentacles. These tentacles grow back within 2 weeks. One may then isolate the nematocysts out of the tentacles by passive dissociation in artificial seawater held at 4° C. overnight. As the tentacle tissue dies and shrinks due to temperature, the nematocysts may be extruded into the solution without firing. As noted above, the product sample here is a mixed population of A-isorhiza, O-isohirza and macro/micro mastigophores.

The nematocytes herein, once loaded with a therapeutic agent as described herein, are preferably attached to a polymeric substrate which can be used as a transdermal substrate material. This is preferably contemplated to include polydimethyldisiloxane (PDMS) whose surface can be activated to promote nematocyst cell binding. The polymeric substrate is preferably supplied in gel form, which is reference to the feature that the polymer can swell in selected solvents. Such polymer gels are preferably cross-linked to augment their ability to reversibly change in volume. Other contemplated examples include poly(acrylamides) and poly(vinyl alcohol), The gels herein are also contemplated to include hydrogels, which are crosslinked hydrophilic polymer systems that swell in water. The above referenced force-firing of the nematocyst can be initiated following attachment of the nematocyst to the PDMS membrane substrate. It is contemplated herein that the nematocyst can be attached to other substrate surfaces, including paper, metals and/or ceramic material.

It is contemplated herein that the loaded nematocysts can be aligned in such a way as to confer a relative uniform directionality during firing. The methods to achieve said directionality herein are contemplated to include the use of magnetic particles, such as iron oxide nanoparticles, for use in magnetic alignment. The level of iron oxide particles in the nematocysts is preferably in the range of 1 microgram per milliliter (1 μg/ml) to 1 picogram per milliliter (1 μg/ml). Upon application of a magnetic force, the nematocysts containing such levels of iron oxide may then be aligned in the magnetic field.

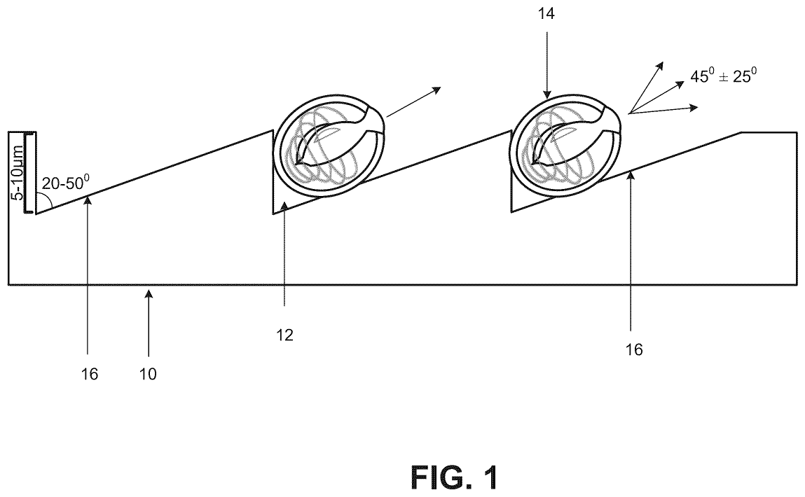

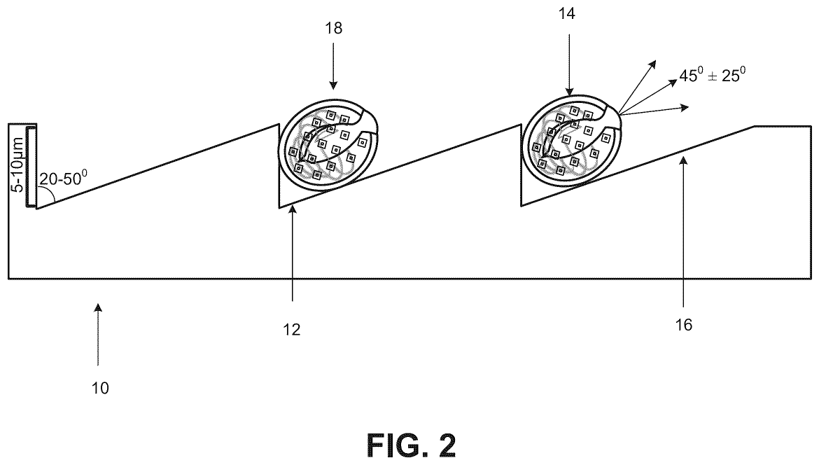

Directionality may also be provided via the use of a 3D-printed mold to imprint a stair-step scaffold onto a surface of a polymeric substrate, which preferably is a polymer gel matrix, to generate topological grooves for the nematocysts to lie in. Attention is therefore directed to FIG. 1 which provides a cross-section of an imprinted poly(dimethylsiloxane) or PDMS polymer gel matrix 10 of a staircase type array, showing separated inclined grooves 12 to contain the nematocysts 14 where the grooves include inclined bottom surfaces 16. As can be seen, the bottom surface incline of the groove is preferably in the range of 20° to 50° from normal and the depth is preferably 5-10 μm. As can be seen, the nematocysts are contemplated to fire with a selected directionality. As illustrated, the directionality is at a selected angle (in this case 45 °) with a variation of +/−25°. Accordingly, the present disclosure provides a polymer gel matrix with a plurality of grooves having an inclined bottom surface for the nematocysts to engage, which then provides that the nematocysts fire a drug and/or vaccine type deliverables (discussed more fully herein) in a selected direction. Or stated another way, the nematocysts are configured to rest within grooves within an imprinted polymer gel matrix such that their payloads (drugs and/or vaccine type deliverables), upon firing, wind-up as concentrated in a desired direction or to a desired location. FIG. 2 is an image similar to FIG. 1 which shows the optional use of magnetic nanoparticles 18 to augment the ability to control the firing of the nematocysts in a desired direction or to a desired location.

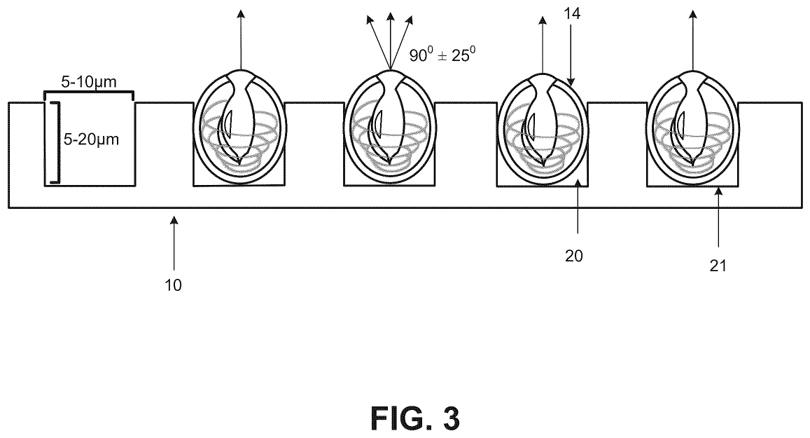

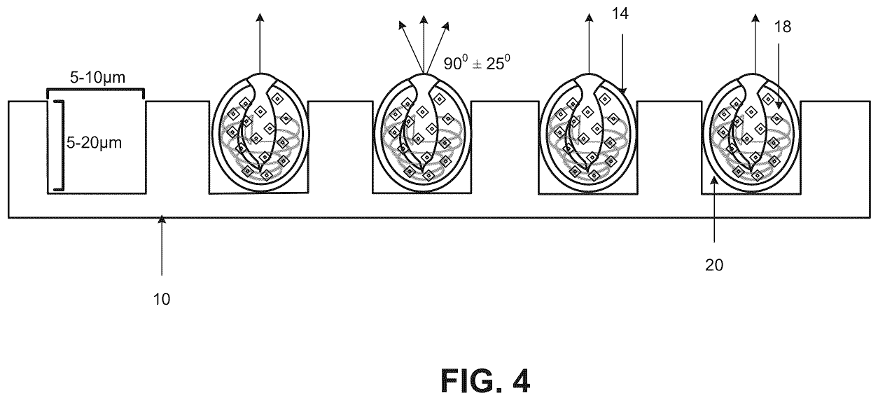

FIG. 3 provides a cross-sectional view of an imprinted polymer gel matrix 10 having recesses or indentations 20 for the nematocysts 14. As can again be observed, the nematocysts may be partially or fully contained within the recesses or indentations 20 and are again configured to fire with a desired directionality. As shown, the recesses or indentations have a relatively flat and non-inclined bottom surface 21. As shown in FIG. 3, the directionality is 90°+/−25°. The recesses or indentations 20 preferably have a depth of 5.0 μm to 20 μm and a preferred width of 5.0 μm to 10.0 μm. FIG. 4 is similar to FIG. 3, again showing the optional use of nanoparticles 18 to augment the ability to control the firing of the nematocysts in a desired direction.

Functional establishment of a directionally aligned nematocyst system is contemplated to be determined by light microscopy and determination of the angle of discharge relative to the bottom of the polymeric gel.

As alluded to above, the nematocysts, after force-firing, can be loaded with a therapeutic agent, such as drugs and/or vaccine type deliverables. Reference to a drug includes biologically active agents such as anti-biotic agents, anti-fungal agents, non-steroidal anti-inflammatory drugs, immunosuppressants, anti-histamine agents, etc. The drug may include a pro-drug, which is activatable prior to, during or following the discharge from the nematocysts. Vaccine type deliverables include vaccines and anti-viral agents.

The molecules contemplated for loading herein include molecules that preferably have a size in the range of up to 2.0 μm. Additional preferred examples of drugs include but is not limited to stimulants (e.g. caffeine), monoclonal antibodies (e.g. Rituximab), and/or small molecule inhibitors (e.g. Oseltamivir). Examples of prophylactics include vaccine antigens, such as virus surface proteins (e.g. haemagglutinin of influenza), or bacterial polysaccharide protein components (e. g factor H-binding protein, fHbp, or meningococcus).

It is further contemplated that the loaded nematocytes herein, providing what may also be described as a needle-free delivery system, will provide relatively increased stability and shelf life of the deliverables (e.g., drugs, vaccines and/or proteins). It is contemplated that shelf life of the aforementioned deliverables within the loaded nematocysts may fall in the range of days to years. Such shelf life may therefore preferably fall in the range of 30 days to 730 days. The loaded nematocytes therefore will provide an alternative to existing intraperitoneal, intradermal, intrathecal, or intramuscular delivery protocols. The depth (a preferred maximum of 800 μm) of therapeutic delivery is directly linked to type of nematocyst and the species of isolation, as these are determinant factors impacting filament length and discharge.

1. A method of providing nematocysts for a transdermal or cutaneous delivery comprising:
a. providing nematocysts sourced from the genus Cassiopea or Chrysaora containing enodgenous toxins;
b. force-firing said nematocysts and collecting said endogenous toxins;
c. treating said nematocysts with an enzyme and inactivating said endogenous toxins.

2. The method of claim 1 wherein said nematocysts sourced from Cassiopea comprises O-izohiza.

3. The method of claim 1 wherein said nematocysts sourced from Chrysaora comprise A-isorhiza, O-isohiza or mastigophores.

4. The method of claim 1, wherein subsequent to force-firing of said nematocysts the nematocysts are loaded with a drug, prophylactic, or vaccine type deliverable.

5. The method of claim 4 wherein said loaded nematocysts are attached to a polymeric substrate.

6. The method of claim 5 wherein said polymeric substrate includes a plurality of grooves having a bottom inclined surface and wherein at least a portion of said loaded nematocysts are contained in said grooves.

7. The method of claim 6 wherein said polymeric substrate includes a plurality of recesses having a relatively flat bottom surface and wherein at least a portion of said loaded nematocysts are contained in said recesses.

8. The method of claim 4 wherein said loaded nematocysts are combined with magnetic particles.

9. The method of claim 5 wherein said polymeric substrate comprises a hydrogel.

10. The method of claim 5 wherein said polymeric substrate comprises a polydimethylsiloxane, poly(acrylamides) or poly(vinyl alcohol).

11. A method of providing nematocysts for a transdermal or cutaneous delivery comprising:
a. providing nematocysts sourced from the genus Cassiopea or Chrysaora containing enodgenous toxins;
b. treating said nematocysts with an enzyme configured to inactivate said endogenous toxins;
c. force-firing said nematocysts and inactivating said endogenous toxins.

12. The method of claim 11 wherein said nematocysts sourced from Cassiopea comprises O-izohiza.

13. The method of claim 11 wherein said nematocysts sourced from Chrysaora comprise A-isorhiza, O-isohiza or mastigophores.

14. The method of claim 11, wherein subsequent to force-firing of said nematocysts the nematocysts are loaded with a drug, prophylactic, or vaccine type deliverable.

15. The method of claim 14 wherein said loaded nematocysts are attached to a polymeric substrate.

16. The method of claim 15 wherein said polymeric substrate includes a plurality of grooves having a bottom inclined surface and wherein at least a portion of said loaded nematocysts are contained in said grooves.

17. The method of claim 16 wherein said polymeric substrate includes a plurality of recesses having a relatively flat bottom surface and wherein at least a portion of said loaded nematocysts are contained in said recesses.

18. The method of claim 15 wherein said loaded nematocysts are combined with magnetic particles.

19. The method of claim 15 wherein said polymeric substrate comprises a hydrogel.

20. The method of claim 15 wherein said polymeric substrate comprises a polydimethylsiloxane, poly(acrylamides) or poly(vinyl alcohol).

21. A pharmaceutical composition comprising as an active ingredient, a therapeutic agent loaded into nematocysts sourced from the genus Cassiopea or Chrysaora.

22. The pharmaceutical composition of claim 19 wherein said nematocysts sourced from Cassiopea comprises O-izohiza.

23. The pharmaceutical composition of claim 19 wherein said nematocysts sourced from Chrysaora comprise A-isorhiza, O-isohiza or mastigophores.

24. The pharmaceutical composition of claim 19 wherein said pharmaceutical composition is combined with magnetic particles which are then subject to a magnetic field and said nematocysts are aligned in a selected direction.

25. The pharmaceutical composition of claim 19 wherein said pharmaceutical composition is placed in a mold having grooves that receive said nematocysts.

26. The pharmaceutical composition of claim 19, wherein said therapeutic agent comprises a drug, prophylactic, or vaccine type deliverable.

27. The pharmaceutical composition of claim 19 wherein said pharmaceutical composition is attached to a polymeric substrate.

28. The pharmaceutical composition of claim 19 wherein said pharmaceutical composition is attached to a hydrogel.

29. The pharmaceutical composition of claim 19 wherein said polymeric substrate comprises a polydimethylsiloxane, poly(acrylamides) or poly(vinyl alcohol).
